(19) United States
(12) Patent Application Publication
(10) Pub. No.: US 2024/0102055 A1
(43) Pub. Date: Mar. 28, 2024

(54) METHOD FOR PRODUCING A FERMENTATION PRODUCT

(71) Applicant: CALIDRIS BIO, Antwerpen (BE)

(72) Inventors: Brecht Marcel VANLERBERGHE, Gent (Sint-Amandsberg) (BE); Frederik Benoit Alphonsine DE BRUYN, Gent (BE); Lieve Monique Cornelia HOFLACK, Zottegem (BE); Johan Hilaire Corneel GHEERAERT, Poperinge (BE)

(21) Appl. No.: 18/265,891

(22) PCT Filed: Dec. 8, 2021
(86) PCT No.: PCT/EP2021/084781

§ 371 Date: Jun. 7, 2023

(30) Foreign Application Priority Data
Dec. 8, 2020 (EP) 20212569.6

(51) Int. Cl. C12P 5/02; C12M 1/00; C12N 1/20

(52) U.S. Cl. CPC C12P 5/023; C12M 29/06; C12M 29/24; C12M 47/10; C12N 1/20

(57) ABSTRACT

The present invention relates to a method for producing a fermentation product, which method comprises (i) reducing carbon dioxide to a C1 compound, (ii) contacting at least a portion of said C1 compound with a culture comprising a methylotrophic microorganism, (iii) fermenting said C1 compound with said methylotrophic microorganism to produce said fermentation product, wherein the fermentation of the C1 compound with said methylotrophic microorganism further produces carbon dioxide, which is at least partially recycled to the reducing step.

### CROSS-REFERENCE TO RELATED APPLICATIONS

This application is a U.S. National Phase Patent Application and claims priority to and the benefit of International Patent Application No. PCT/EP2021/084781, filed on Dec. 8, 2021, which claims priority to European Patent Application No. 20212569.6, filed on Dec. 8, 2020. The entire contents of both of which are incorporated herein by reference.

### FIELD OF THE INVENTION

The present invention relates to methods for producing a fermentation product, which method comprises (i) reducing carbon dioxide to a C1 compound, (ii) contacting at least a portion of said C1 compound with a culture comprising a methylotrophic microorganism, (iii) fermenting said C1 compound with said methylotrophic microorganism to produce said fermentation product, wherein the fermentation of the C1 compound with said methylotrophic microorganism further produces carbon dioxide, which is at least partially recycled to the reducing step.

### BACKGROUND OF THE INVENTION

High value organic compounds such as amino acids, proteins and vitamins are used in a broad range of applications.

Lately, such organic compounds have mainly been derived from agricultural, or fossil sources. However, increased pressure has nowadays been placed on traditional fossil hydrocarbon inputs and there is also an urging need to reduce the amount of greenhouse gases emissions to the atmosphere, as well as global energy consumption in chemical production systems. For this reason, chemical engineers are exploring and adopting methods that use renewable energy for the conversion of carbon dioxide, or other low value carbon sources into useful organic chemicals.

While some effort has been directed to fully abiotic and chemical processes for carbon dioxide conversion, most of the focus in the area has been placed on biological processes that fix carbon dioxide into biomass and/or valuable end-products. Photosynthetic organisms, such as plants, algae and cyanobacteria can be used to this end. However, these organisms rely on the relatively inefficient process of photosynthesis to supply the energy needed for production of organic compounds from carbon dioxide. Moreover, commercial production of organic chemicals using photosynthetic organisms requires reliable and consistent exposure to light in order to achieve high productivity.

A first alternative to the use of photosynthetic organisms is to make use of microorganisms which are capable of directly fermenting gaseous streams comprising H₂ and CO₂. In most cases, such industrial processes start from synthetic gas “syngas” as substrate, which is a combination of varying amounts of H₂, CO and CO₂ frequently derived from gasified coal or natural gas.

For example, WO 2017/136478 and WO 2019/204029 describe the direct fermentation of syngas to multiple products such as alcohols, olefins or lipids. However, syngas often contains impurities which must be removed by complex and costly purification processes before the gas can be provided to the methylotrophic microorganisms.

An further alternative to the use of photosynthetic organisms is to first convert carbon dioxide into C1 compounds (containing no carbon-carbon bonds) such as methanol, formate, formic acid, formaldehyde or methane, which C1 compounds can then be used by methylotrophic microorganisms as energy and/or carbon source to produce more complex organic compounds. The conversion of carbon dioxide to C1 compounds, such as methanol, presents certain benefits over the conventional process, both from an economic and an environmental point of view. Starting from pure carbon dioxide and a separate source of pure hydrogen, rather than a mixture of CO, CO₂ and H₂ as is the case with syngas, simplifies the chemistry and reaction products. In addition, diverting carbon dioxide from the atmosphere and into C1 compounds offers the possibility to recycle large quantities of atmospheric carbon dioxide (Alvarado et al., 2016, HIS Chem. Bull., 3, 10-11).

WO 2015/021352, WO 2014/012055 and WO 2014/089436 demonstrate the potential of methylotrophic microorganisms for the fermentative production of several organic compounds. There is therefore a growing interest for the development of effective integrated systems using methylotrophic microorganisms to ferment C1 compounds in order to produce high value organic chemicals on commercially-relevant scale, and this for an acceptable price. Yet, improving the efficiency of industrial systems producing high value organic compounds by microbial fermentation of C1 compounds remains a challenge. Most processes focus on the optimization of the fermentative process through e.g. the use of engineered and/or evolved methylotrophic microorganisms. Still, known systems do not solve the root issue of the sustainable and energy-efficient sourcing of the C1 substrate.

While microbiological conversion of carbon dioxide to valuable fermentation products has tremendous benefits for the environment, improvements in efficiency and cost reductions are still needed to make these processes economically viable. In view of the above there is a continuing need to develop improved, more efficient, and sustainable methods for large-scale fermentative production of high value organic chemicals, especially methods that reduce the emission of greenhouse gases such as carbon dioxide.

### SUMMARY OF THE INVENTION

The inventors have identified methods to improve the efficiency of the conversion of carbon dioxide to fermentation products. The present invention provides integrated processes and apparatuses for carbon dioxide conversion. In a first aspect, the present invention provides a method for producing a fermentation product, which method comprises:
reducing carbon dioxide to a C1 compound,
contacting at least a portion of said C1 compound with a culture comprising a methylotrophic microorganism,
fermenting said C1 compound with said methylotrophic microorganism to produce said fermentation product,
wherein the fermentation of the C1 compound with said methylotrophic microorganism further produces carbon dioxide, which is at least partially recovered and recycled to the reducing step.

It has further been found that oxygen released during the carbon dioxide reduction can be utilized in the fermentation process. Therefore, in a particular embodiment, the present invention provides the above method wherein the carbon dioxide reduction process co-generates oxygen and wherein the oxygen is used to maintain aerobic conditions in the fermentation process.

In one particular embodiment, the carbon dioxide reduction is performed by carbon dioxide hydrogenation. Preferably, water electrolysis is used to generate hydrogen for the hydrogenation and oxygen that is utilized in the fermentation process. This provides an integrated system with improved utilization of source materials and a lowered environmental impact. Therefore, in a further embodiment, the present invention provide a method as described herein, wherein the carbon dioxide reduction process comprises electrolysis of water to oxygen and hydrogen, wherein the hydrogen is used for hydrogenation of carbon dioxide and the oxygen is used to maintain aerobic conditions in the fermentation process.

In another particular embodiment of the invention, the carbon dioxide reduction process comprises electrochemical reduction of carbon dioxide.

In yet another particular embodiment, said C1 compound is soluble in water. Exemplary C1 compounds soluble in water for use in the methods and of the invention include methanol, formaldehyde, formic acid, and formate, and combinations thereof. In a preferred embodiment, said C1 compound is methanol.

Therefore, in a particular embodiment, the method of the invention comprises electrolysis of water to hydrogen and oxygen, wherein
the hydrogen is used for the hydrogenation of carbon dioxide to methanol, and
the oxygen is used to maintain aerobic conditions during the fermentation of the methanol with the methylotrophic microorganism; and
wherein the fermentation produces carbon dioxide which is at least partially recovered and recycled to the hydrogenation process.

Therefore, in another particular embodiment, the method of the invention comprises electrochemical reduction of carbon dioxide to methanol, wherein oxygen is co-generated, and wherein the oxygen is used to maintain aerobic conditions during the fermentation of the methanol with the methylotrophic microorganism; and
wherein the fermentation produces carbon dioxide which is at least partially recovered and recycled to the electrolysis process.

It has furthermore been identified that synergistic efficiency improvements can be obtained by performing the fermentation as well as the recovering of carbon dioxide from the fermentation process at increased pressures. In particular, increased pressures improve fermentation efficiency as well as the efficiency of carbon capture from the fermentation process. Therefore, in a particular embodiment of the invention, the fermenting as well as the recovering of carbon dioxide is performed at an increased pressure.

The methods of the invention are broadly applicable and can be used with methylotrophic microorganisms in general and, preferably, aerobic methylotrophic microorganisms. In a particular embodiment, said methylotrophic microorganism is a microorganism selected from the group consisting of Methylomonas, Methylobacter, Methylococcus, Methylosinus, Methylocyctis, Methylomicrobium, Methanomonas, Methylophilus, Methylobacillus, Methylobacterium, Hyphomicrobium, Xanthobacter; Bacillus, Paracoccus, Nocardia, Arthrobacter, Rhodopseudomonas, Pseudomonas, Candida, Hansenula, Pichia, Torulopsis, and Rhodotorula.

In another particular embodiment, said fermentation product comprises a carbon backbone that is five carbons or longer. In a further embodiment, said fermentation product is selected from an enzyme, an antibiotic, an amino acid, a protein, a plant biostimulant, a growth enhancer, a probiotic, a prebiotic, a biofertilizer, a food, a feed, a vitamin, a lipid, a bioplastic, a polysaccharide, biomass, a bioceutical or a pharmaceutical. In a further embodiment said fermentation product is a protein. Advantageously, the fermentation product may be a proteinaceous biomass, such as a proteinaceous biomass that can serve as a protein source in food or feed products, e.g. single cell protein (SCP).

In another aspect, the invention provides a system for performing the methods of the invention. In a further embodiment, the invention provides an apparatus for performing the methods of the invention. Therefore, in a particular embodiment, the present invention provides a system comprising:
a reduction vessel for reducing carbon dioxide to a C1 compound;
a fermentation vessel for culturing a methylotrophic microorganism to convert the C1 compound to a fermentation product; and
a carbon dioxide recovering unit connected to said fermentation vessels to recover at least part of the carbon dioxide produced in the fermentation vessel.

In another embodiment, the present invention provides an apparatus for producing a fermentation product according to the method of any one of the preceding claims comprising:
a reduction vessel for reducing carbon dioxide to a C1 compound;
a fermentation vessel for culturing a methylotrophic microorganism and connected to said first vessel via a duct that allows to transport the C1 compound to the fermentation vessel;
a carbon dioxide recovering unit connected to said reduction and fermentation vessels that allows to recover at least part of the carbon dioxide produced in the fermentation vessel and to recycle said recovered carbon dioxide to the reduction vessel.

In a further embodiment, the apparatus further comprises an electrolysis vessel for electrolysis of water, wherein the electrolysis vessel comprises a duct to transport hydrogen generated from water electrolysis to the reduction vessel, and a duct to transport oxygen generated from water electrolysis to the fermentation vessel.

### BRIEF DESCRIPTION OF THE DRAWINGS

FIG. 1: Schematic overview of an embodiment of the process of the invention, comprising the reduction of exogenous CO₂ with electricity to produce a reduced C1 compound (C1_{red}) that is entered in a fermentation vessel. Oxygen (O₂) from the CO₂ reduction process is added to the fermentation vessel to provide an aerobic fermentation environment for the generation of a fermentation product. CO₂ from fermentation exhaust gasses (endogenous CO₂) is captured and recycled to the CO₂ reduction vessel.

FIG. 2 Schematic overview of a specific embodiment of the process of the invention, comprising the electrolysis of water to produce hydrogen and oxygen, wherein the hydrogen is used for the hydrogenation of CO₂ and oxygen is used for providing an aerobic fermentation environment in the fermentation vessel.

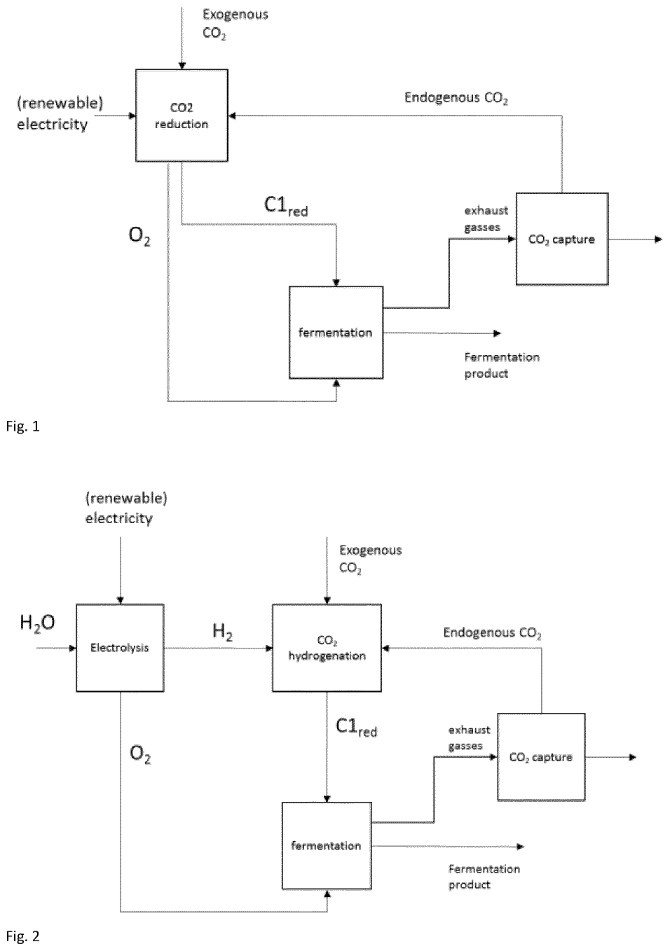

### DETAILED DESCRIPTION OF THE INVENTION

As described herein before, the present invention provides a method for producing a fermentation product, which method comprises:
reducing carbon dioxide to a C1 compound,
contacting at least a portion of said C1 compound with a culture comprising a methylotrophic microorganism,
fermenting said C1 compound with said methylotrophic microorganism to produce said fermentation product,
wherein the fermentation of the C1 compound with said methylotrophic microorganism further produces carbon dioxide, which is at least partially recovered and recycled to the reducing step.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclature used in connection with, and techniques of, biology and chemistry described herein are those well-known and commonly used in the art.

### Carbon Dioxide Reduction

Carbon dioxide reduction methods that reduce carbon dioxide to a C1 compound are known in the field and include carbon dioxide hydrogenation and photochemical, photoelectrochemical and electrochemical CO₂ reduction, which can be used for the methods of the present invention.

By the term “C1 compound”, is meant in the sense of the present invention, a compound which contains only one carbon atom. Examples include but are not limited to formate, formic acid, formamide, formaldehyde, carbon monoxide, methane, methanol, methylamine, halogenated methanes, and monomethyl sulfate. In various embodiments, the invention provides for the C1 compound serving as a source of both energy and carbon for the organism. In one embodiment, the C1 compound is soluble in water. In a further embodiment, the C1 compound is miscible in water. For example, the C1 compound can be methanol, formaldehyde, formic acid, and formate and combinations thereof. C1 compounds that dissolve at high concentration or are miscible in water, in some instances, are preferable to less soluble or immiscible chemical species, such as methane, because mass transfer and uptake by the organism is more efficient. Similarly, water soluble C1 compounds are preferable to molecular hydrogen, carbon dioxide or carbon monoxide, used in fermentative production of carbon-based compounds using knallgas or oxyhydrogen microorganisms. In some embodiments, the C1 compound can be soluble in other solvents than water, depending on the composition of the media used for growing the organism. For example, the solubility of the C1 compound in the media may be enhanced by other components therein. In a preferred embodiment, the C1 compound comprises methanol.

Unless specified otherwise or clear from its context, percentages as used herein refer to weight to weight percentages.

In a particular embodiment, the reduction of carbon dioxide comprises the hydrogenation of carbon dioxide to a C1 compound. As will be understood by the skilled person, the process can be direct hydrogenation of carbon dioxide into the desired C1 compound or the hydrogenation can proceed through a multistep reaction to obtain the desired C1 compound. For example, hydrogenation of CO₂ to methanol (CTM) can be performed through the formation of CO that is further hydrogenated to methanol or through the formation of formate that is further hydrogenated to methanol. An overview of CO₂ hydrogenation methods is for example provided in Ye et al. (Nature communications 2019:5698).

When the reduction of CO₂ to the C1 compound comprises hydrogenation, a preferred source of hydrogen is the electrolysis of water. Therefore, in a particular embodiment, the reduction of carbon dioxide comprises the electrolysis of water to hydrogen (H₂) and oxygen (O₂), wherein the hydrogen is used to hydrogenate the carbon dioxide to a C1 compound. In a further particular embodiment, the electrical energy for the electrolysis comes from a renewable energy source such as but not limited to wind-energy, solar energy, tidal, hydropower and geothermal energy. The inventors have further found that oxygen produced during water hydrolysis can conveniently be used to maintain aerobic conditions during fermentation. Therefore, in a further embodiment, the present invention provides a method as described herein, wherein the carbon dioxide reduction process comprises electrolysis of water to oxygen and hydrogen, wherein the hydrogen is used for hydrogenation of carbon dioxide and the oxygen is used to maintain aerobic conditions in the fermentation process.

As mentioned above, the reduction of carbon dioxide may comprise the electrochemical reduction of carbon dioxide to a C1 compound. The electrochemical production of formate and formic acid from carbon dioxide is for example disclosed in WO2007/041872. Electrochemical reduction of carbon dioxide to formaldehyde and methanol is disclosed in, for example, WO2010/088524, WO2012/015909 and WO2012/015905. The electrochemical reduction of carbon dioxide to methanol is disclosed in WO2006/113293. All these patent references are herein incorporated by reference.

Reduction of carbon dioxide to a C1 compound may produce mixtures of C1 compounds. Therefore, the C1 compounds for use in the invention may comprise mixtures of different C1 compounds as provided herein. Conveniently, it has been observed that mixtures of C1 compounds do not prevent fermentative conversion into fermentation products. It will be further understood that by reducing carbon dioxide to a certain compound, does not exclude that part of the carbon dioxide is reduced in the reduction process to different compounds as well. For example, if it is stated that carbon dioxide is reduced to methanol, this includes the possibility that the only C1 compound resulting from the reduction of carbon dioxide is methanol as well as the possibility that carbon dioxide is partly reduced to methanol and partly to different C1 compounds, such as formic acid, formate, and/or formaldehyde. In a particular embodiment, the majority of the C1 compounds obtained from the reduction of carbon dioxide is a particular or preferred C1 compound as described herein. For example, when pertaining to methanol, this means that the majority of C1 compounds that originate from the reduction of carbon dioxide is methanol, while a minority part may consist of other C1 compounds. In a further particular embodiment, more than 55%, in particular more than 60%, more in particular more than 65% of the C1 compounds that originate from the reduction of carbon dioxide is a particular or preferred C1 compound(s) as described herein. In an even further embodiment, more than 65%, more than 70%, more than 75%, more than 80% of the C1 compounds that originate from the reduction of carbon dioxide is a particular or preferred C1 compound(s) as described herein. In another further embodiment, the C1 compounds that originate from the reduction of carbon dioxide consist essentially of the particular or preferred C1 compound(s) as described herein. In the context of the invention, consisting essentially of a particular or preferred C1 compound, refers to at least 85% of the particular or preferred compound. In particular at least 90%, 92%, 95%, 96%, or 97%.

Preferably, the C1 compound is soluble, preferably miscible, in water. In a particular embodiment, the carbon dioxide reduction is performed in an aqueous liquid. In another particular embodiment, the carbon dioxide reduction generated an aqueous liquid comprising the C1 compound, in particular comprising the C1 compound that is soluble, preferably miscible, in the aqueous liquid. A liquid, preferably an aqueous liquid, comprising the C1 compound can be added to the fermentation process. It has furthermore been found that the fermentation process does not require pure C1 compounds and can ferment exit streams from the carbon dioxide reduction process that have not been purified or have only been purified to a limited extend. This is especially convenient as several carbon dioxide reduction processes have an incomplete efficiency and/or may result in different chemical impurities. Having minor or no purification simplifies the process, allows for the utilization of carbon dioxide reduction processes that have a lower efficiency, reduces energy and resource consumption, and reduces waste. In some embodiments, the C1 compound is not purified to an extent that it is more than 5% w/w, in particular more than 10% w/w, more in particular more than 15% w/w, even more in particular more than 20% w/w. In some embodiment more than 25% w/w, 35% w/w, 50% w/w, 60% w/w, 70% w/w, 80% w/w, 85% w/w, 90% w/w, 95% w/w.

Independent of the carbon dioxide reduction method chosen for use in the present invention, the reduction process co-generates oxygen. In a particular embodiment, the oxygen co-generated during the carbon dioxide reduction process is utilized to maintain aerobic conditions during fermentation. The oxygen co-generated during reduction may be in a relatively pure form, e.g. when derived from water electrolysis, or it may be a non-pure form. Regardless, it has been found that exit streams of the reduction process that are enriched in oxygen co-generated during carbon dioxide reduction are useful to maintain aerobic conditions during fermentation, thereby improving efficiency and reducing variability and raw material source input.

The available external source of carbon dioxide is preferably an exhaust stream from a fossil fuel burning power or other industrial plants, a natural source accompanying natural gas or a biogenic source such as brewing, or biomass treatment options such as anaerobic digestion, pyrolysis, torrefaction, combustion. These available sources would otherwise be released into the atmosphere. The utilization of the exhaust stream as a source for chemical recycling avoids emitting the carbon dioxide into the atmosphere. The available source of carbon dioxide may also be the air of our atmosphere. In a particular embodiment, the carbon dioxide is obtained from the air of the atmosphere by absorbing atmospheric carbon dioxide onto a suitable adsorbent followed by the release of the adsorbed carbon dioxide therefrom, e.g. by heat or pressure treatment. Removing and recycling carbon dioxide from the atmosphere provides a source that is inexhaustible. In a preferred embodiment, the carbon dioxide source comprises carbon dioxide in a concentration that is higher than the concentration of carbon dioxide in the atmosphere. In particular, the carbon dioxide source may comprise more than 1000 ppm of carbon dioxide, such as more than 5.000 ppm or more than 10.000 ppm. In a further preferred embodiment, the % w/w of carbon dioxide in the carbon dioxide source is at least 60%, in particular at least 70%.

Separation of carbon dioxide from other constituents in liquid form may involve liquifying a gas comprising carbon dioxide by compression, cooling and expansion steps. When in liquid form, the carbon dioxide can be separated by distillation. Refrigerated systems may also be used for carbon dioxide separation. For example, carbon dioxide can be recovered from crude syngas produced from gasification or from a stream resulting from reacting the carbon monoxide with steam in a water gas shift reaction to produce a stream comprising carbon dioxide and hydrogen. Further, the biogenic carbon dioxide may be collected from excess carbon dioxide generated during the gasification or collected from a recycle stream, such as, without limitation, a carbon dioxide stream recycled during syngas fermentation. Without being limiting, the carbon dioxide can be separated by physical or chemical absorption to produce a carbon dioxide-containing stream. The physical absorption may involve the use of membranes that allow the selective permeation of a gas through them. For example, the carbon dioxide can be recovered by membranes that are more permeable to carbon dioxide than other components in the carbon dioxide-containing stream. The carbon dioxide passes through the membrane while other components do not, thereby resulting in a stream that is carbon dioxide enriched. The carbon dioxide-enriched stream can be used in gas or liquid form. Chemical absorption involves the use of chemical solvents. Examples of chemical solvents include methanol, N-methyl-2-pyrolidone, dimethyl ethers of polyethylene glycol, potassium carbonate, monoethanolamine, methyldiethylamine and tetrahydrothiophene 1,1-dioxide. Amine gas scrubbing is another example of a technique involving chemical absorption. A prevalent amine for such applications in monethanolamine. Carbon dioxide can be obtained from a gaseous stream, such as a flue gas stream produced from a combustion process that uses the non-fossil organic material as a feed. This includes combustion of organic material in a power plant, such as a plant that otherwise burns fossil fuel such as natural gas or coal. Such a combustion includes an oxyfuel combustion process. Gaseous streams from combustion contain carbon dioxide and other impurities depending on the source. Carbon dioxide can be separated from impurities in the gas stream using a liquid absorbent or solid sorbent that is capable of capturing carbon dioxide. The liquid absorbent may be a chemical solvent, such as an amine, or a Selexol™ solvent which uses polyethylene glycol as a solvent. The liquid absorbent can be added as part of a scrubbing operation, such as amine scrubbing. Regeneration of the chemical solvent may then be conducted by stripping or other separation techniques, with the regenerated chemical solvent being used to capture more carbon dioxide. A solid sorbent may include a zeolite or activated carbon. For solid sorbents, regeneration may be achieved by a change in pressure or temperature, thereby releasing the carbon dioxide and regenerating the sorbent for further use. Biogenic carbon dioxide from oxyfuel combustion can be separated from other gaseous components by distillation. A carbon dioxide-containing stream can be liquefied by compression, cooling and expansion steps. The carbon dioxide can subsequently be separated in liquid form in a distillation column. A further example of a technique for carbon dioxide separation from other components is refrigerated separation. Distillation or refrigerated separation can also be used to separate carbon dioxide from synthesis gas that has undergone a water-gas shift conversion of carbon monoxide to carbon dioxide.

### Fermentation

By the term «fermenting», is meant in the sense of the present invention the conversion of inorganic or organic carbon-based substrates through the enzymatic processes of microorganisms into fermentation products. Fermentation can be aerobic or anaerobic. By the term «fermentation product», is meant in the sense of the present invention, organic compounds of interest obtained by fermentation. Fermentation products include but are not limited to alcohols, fatty acids, fatty acid derivatives, fatty alcohols, fatty acid esters, wax esters, hydrocarbons, alkanes, polymers, amino acids, proteins, fuels, commodity chemicals, specialty chemicals, carotenoids, isoprenoids, sugars, sugar phosphates, central metabolites, pharmaceuticals, and pharmaceutical intermediates. For example, the fermentation products can include one or more sugars (for example glucose, sucrose, xylose, lactose, maltose, pentose, rhamnose, galactose, or arabinose), sugar phosphate (for example glucose-6-phosphate, or fructose-6-phosphate), sugar alcohol (for example sorbitol), sugar derivative (for example ascorbate), alcohol (for example ethanol, propanol, isopropanol, or butanol), ethylene, propylene, 1-butene, 1,3-butadiene, acrylic acid, fatty acid (for example co-cyclic fatty acid), fatty acid intermediate or derivative (for example fatty acid alcohol, fatty acid ester, alkane, olefin, or halogenated fatty acid), amino acid or intermediate (for example, lysine, glutamate, aspartate, shikimate, chorismate, phenylalanine, tyrosine, tryptophan), phenylpropanoid, isoprenoid (for example hemiterpene, monoterpene, sesquiterpene, triterpene, polyterpene, isoprene, carotene, lycopene, limonene or polyisoprene), glycerol, 1,3-propanediol, 1,4-butanediol, 1,3-butadiene, polyhydroxyalkanoate, polyhydroxybutyrate, acrylate. The fermentation product can also be biomass. In a particular embodiment, the fermentation product is an amino acid, a protein or a vitamin. In a more particular embodiment, the fermentation product is a protein. In a further particular embodiment, the fermentation product is a biomass with a high protein content, more particularly a biomass with a high protein content that is suitable for use as a protein source in animal feed. In another particular embodiment, the fermentation product is a biomass with a high protein content that is suitable for use as a protein source in human food.

In a particular embodiment, the fermentation product is single cell protein (SCP). “Single cell protein” or “microbial protein” refers to a protein derived from organisms that exist in the unicellular, or single cell, state. This includes unicellular bacteria, yeasts, fungi or eukaryotic single cell organisms such as algae. The SCP has many uses, including uses as food and animal feed. Microbes often employed for the production of SCP include yeast (such as Saccharomyces, Pichia, Candida, Torulopsis, and Geotrichum), fungi (such as Aspergillus, Fusarium, Sclertoium, Polyporus, Trichoderma, and Scytalidium), Bacteria (such as Rhodobacter), and Algae (such as Athorspira and Chlorella). SCP derived from fungi (which include yeasts) is also known as mycoprotein (e.g. Quorn).

As used herein, “biomass” or “biological material” refers to organic material having a biological origin, which may include one or more of whole cells, lysed cells, extracellular material, or the like. For example, the material harvested from a cultured microorganism (e.g., bacterial or yeast culture) is considered the biomass, which can include cells, cell membranes, cell cytoplasm, inclusion bodies, products secreted or excreted into the culture medium, or any combination thereof. In certain embodiments, biomass comprises the C1 metabolizing microorganisms of this disclosure together with the media of the culture in which the C1 metabolizing microorganisms of this disclosure were grown. In other embodiments, biomass comprises C1 metabolizing microorganisms (whole or lysed or both) of this disclosure recovered from a culture grown on a C1 substrate (e.g., methanol and/or formic acid). In still other embodiments, biomass comprises the spent media supernatant from a culture of C1 metabolizing microorganism cultured on a C1 substrate. Such a culture may be considered a renewable resource.

By the term “culture”, is meant in the sense of the present invention, a population of unicellular or multicellular microorganisms in a medium, such as a growth or fermentation medium.

By the term “methylotrophic microorganism” or “methylotroph” as used herein refers to any microorganism that is able to use a C1 compound as an energy or carbon source for their growth and development. Methylotrophs often use C1 compounds as both a source of energy and carbon. For example, the methylotrophic microorganism can be chosen from eukaryotic or prokaryotic microorganisms, such as bacteria (Gram-negative (for example Alphaproteobacteria) or Gram-positive), archaea, protist, or fungi. Suitable methylotrophic microorganisms include those which are commonly used in laboratory and/or industrial applications. In a preferred embodiment, the methylotrophic microorganism is a non-photosynthetic microorganism. In another particular embodiment, the methylotrophic microorganism is not an oxyhydrogen microorganisms, also known as a knallgas microorganism or hydrogen oxidizing microorganisms. In particular, the methylotrophic microorganism does not oxidize hydrogen with oxygen as a source of energy.

In some embodiments, host cells/organisms can be selected from the group consisting of Methylomonas, Methylobacter, Methylococcus, Methylosinus, Methylocyctis, Methylomicrobium, Methanomonas, Methylophilus, Methylobacillus, Methylobacterium, Hyphomicrobium, Xanthobacter, Bacillus, Paracoccus, Nocardia, Arthrobacter, Rhodopseudomonas, Pseudomonas, Candida, Hansenula, Pichia, Torulopsis, and Rhodotorula. In certain further embodiments, the bacterium is Methylophilus methylotrophus or Methylobacterium extorquens.

In a further aspect of the invention, the methylotrophic microorganism can be an engineered methylotrophic microorganism. As used herein the term “engineered methylotrophic microorganism” refers to organisms that have been genetically engineered to convert C1 compounds, such as formate, formic acid, formaldehyde or methanol, to fermentative products. As used herein, an engineered methylotrophic microorganism should not necessarily derive its organic carbon solely from C1 compounds. The term engineered methylotrophic microorganism includes originally methylotrophic microorganisms that have been genetically engineered to include one or more energy conversion, carbon fixation, methylotrophic and/or carbon product biosynthetic pathways in addition or instead of its endogenous methylotrophic capability as well as originally non-methylotrophic microorganisms that have been genetically engineered to introduce C1 conversion pathways. The term “engineer”, “engineering” or “engineered”, as used herein, refers to genetic manipulation or modification of biomolecules such as DNA, RNA and/or protein, or similar techniques commonly known in the biotechnology art.

Further, the methylotrophic microorganism can be metabolically evolved, for example for the purposes of optimized energy consumption, methylotrophy and/or carbon fixation. The terms “metabolically evolved” or “metabolic evolution” relates to a growth-based selection of methylotrophic microorganism that demonstrate improved growth.

The engineered and/or evolved methylotrophs of the invention can be produced by introducing expressible nucleic acids encoding one or more of the enzymes or proteins participating in one or more carbon product biosynthetic pathways. Depending on the host methylotroph chosen, nucleic acids for some or all of particular metabolic pathways can be expressed. For example, if a chosen host methylotroph is deficient in one or more enzymes or proteins for desired metabolic pathways, then expressible nucleic acids for the deficient enzyme(s) or protein(s) are introduced into the host for subsequent exogenous expression. Alternatively, if the chosen host methylotroph exhibits endogenous expression of some pathway genes, but is deficient in others, then an encoding nucleic acid is needed for the deficient enzyme(s) or protein(s) to achieve production of desired carbon products from C1 compounds. Thus, an engineered and/or evolved methylotroph of the invention can be produced by introducing exogenous enzyme or protein activities to obtain desired metabolic pathways or desired metabolic pathways can be obtained by introducing one or more exogenous enzyme or protein activities that, together with one or more endogenous enzymes or proteins, produces a desired product such as reduced cofactors, central metabolites and/or carbon-based products of interest. Depending on the metabolic pathway constituents of a selected host methylotroph, the engineered and/or evolved methylotrophs of the invention can include at least one exogenously expressed metabolic pathway-encoding nucleic acid and up to all encoding nucleic acids for one or more energy conversion, carbon fixation, methylotrophic and/or carbon-based product pathways.

The growth of the microorganism is sensitive to the operating temperature of the fermenter and each particular microorganism has an optimum temperature for growth and determining these conditions is well within the ambit of the skilled person. The broad temperature range employed for the fermentation process of this invention would be from about 25° C. to 65° C. and more preferably between 30° and 60° C. The temperature selected will generally depend upon the microorganism employed in the process since they will have a somewhat different temperature/growth rate relationship.

In the practice of the present invention, a suitable nutrient medium may supplied to the fermenter in addition to the C1 compound, e.g. to provide nutrients such as an assimilable source of nitrogen, phosphorus, magnesium, calcium, potassium, sulfur and sodium as well as trace quantities of copper, manganese, molybdenum, zinc, iron, boron, iodine and selenium. As is well known in the art of fermentation, the relative amounts of the above nutrients can vary depending on the microorganism selected for the process. In addition, the nutrient medium can also contain vitamins as is known in the art when their presence is known to be desirable for the propagation of certain microorganisms. For example, many yeasts appear to require the presence of one or both of the vitamins, biotin and thiamin for their proper propagation.

The fermentation reaction is preferably an aerobic process wherein the oxygen needed for the process can be supplied from a free oxygen-containing source such as air which is suitably supplied to the fermentation vessel. One good source of oxygen is oxygen enriched air. A preferred source of oxygen is oxygen enriched air wherein the oxygen is derived from the carbon dioxide reduction process. It is preferred that the oxygen-containing source is admixed with the fermentation culture, e.g. by bubbling the oxygen-containing source, e.g. oxygen enriched air, through the fermentation culture.

The fermentation reaction is found to be favorably affected by use of an increased pressure, i.e. a pressure above the atmospheric pressure. In a particular embodiment, the fermentation is performed at a pressure of 2 bar or more, in particular 5 bar or more, more in particular 10 bar or more. In another particular embodiment, the fermentation is performed in a pressure range of 2-100 bar, in particular 2 to 50 bar, more in particular 2 to 20 bar.

Preferably the fermentation process of the instant invention is a continuous type but it is to be noted that it can be conducted as a batch or fed-batch process. In the continuous, fed-batch or batch process modes of operation the fermentation reactor is first sterilized and subsequently inoculated with a culture of the desired microorganism in the presence of all the required nutrients including oxygen and the carbon source. The oxygen source or air is continuously introduced. In the continuous and fed-batch method of operation there is also a continuous introduction of nutrient medium, nitrogen source (if added separately) and the C1 compound at a rate which is either predetermined or in response to need which can be determined by monitoring such things as C1 concentration, alcohol concentration, dissolved oxygen, and oxygen or carbon dioxide in the gaseous effluent from the fermenter. The feed rate of the various materials can be varied so as to obtain as rapid a cell growth as possible consistent with efficient utilization of the C1 compound feed, i.e., a high yield of cell weight per weight of C1 compound feed charged.

As is known in the art, the feed rate of the C1 compound is an important variable to control since in high concentration this material can actually inhibit cell growth and may even kill the microorganism. This is especially relevant if the C1 compound is methanol as most microorganisms can only sustain limited alcohol percentages. Therefore, the feed rate of the C1 compound is preferably adjusted such that the C1 compound is consumed by the microorganism at essentially the same rate as it is being fed to the fermenter. When this condition is attained there will be, of course, little or no C1 compound in the effluent which is continuously withdrawn from the fermenter in a continuous type of process. However, satisfactory operation can be achieved with up to about 1% v/v of the C1 compound in the effluent. For high cell productivity or growth rate, the concentration of C1 compound in the feed to the fermenter can for example be from about 7 percent up to about 30% v/v.

For batch or continuous operation of the process of this invention, the concentration of C1 feedstock, e.g., methanol, in the fermenter may for example be within the range of from 0.001 up to 5.0% v/v, such as from 0.005 up to 3.0% v/v, or from 0.01 up to 2.0% v/v, and preferably from 0.01 up to 0.5% v/v. It is possible, of course, and may in some instances be desirable, to add the feedstock incrementally to an otherwise typical batch fermentation process. Optionally, in addition to the C1 compound originating from the CO₂ reduction process, an external source of C1 compound may be added to the fermentation vessel. Such C1 compound may e.g. be obtained from a commercial supplier. The addition of an external source of C1 compound may be helpful to reduce the impact of fluctuations in the generation of C1 compound in the CO₂ reduction process.

The fermentation product may be collected from the fermentation culture using methods known in the field of batch or continuous fermentation. As is known in the field, collection of fermentation product will typically depend on the localization of the fermentation product, e.g. intracellular or extracellular of the methylotrophic microorganism. In a particular embodiment, collecting the fermentation product comprises obtaining fermentation culture and at least partially removing methylotrophic microorganisms to obtain fermentation medium comprising the fermentation product. In another particular embodiment, collecting the fermentation product comprises obtaining fermentation culture and concentrating methylotrophic microorganisms therein to obtain a concentrated product comprising the fermentation product. In a further particular embodiment, removing or concentrating methylotrophic microorganisms is performed by filtration or centrifugation, with filtration being preferred. Obtaining the fermentation product may require further processing, such as further purification or enrichment steps, and washing or heat treatment steps. In continuous fermentation, portions of the fermentation culture that are not withheld for collection of the fermentation product may be rerouted to the fermentation vessel.

In one preferred embodiment, the fermentation product is single cell protein, herein also referred to as a biomass with a high protein content. In such embodiment, the fermentation culture may be filtrated and/or centrifuged to concentrate the methylotrophic microorganisms. The portion that is not withheld (filtration pass-through or supernatant) may be returned to the fermentation vessel for continued fermentation. An additional step might be needed to sterilize the fermentation product. The concentrated biomass may undergo further washing, heat treatment, formulation and drying steps to obtain single cell protein that is suitable for animal feed or human food consumption. In a particular embodiment, the fermentation product is combined with one or more additional ingredients, such as a free-flow agent or an anti-oxidant.

In one particular embodiment, the present invention further provides the use of a fermentation product of the invention as an animal feed product. In another embodiment, the present invention provides an animal feed product comprising a fermentation product of the invention. In a further embodiment, the present invention provides an animal feed product of the invention wherein at least 10%, in particular at least 20%, more in particular at least 30% of the protein content is derived from the fermentation product of the invention. More in particular at least 40%, 50%, 60%, or 70%. In a preferred embodiment, at least 80%, more preferably at least 90%. The present invention further provides methods for producing an animal feed product, the method comprising mixing animal feed ingredients with the fermentation product of the invention to obtain an animal feed product.

In another particular embodiment, the present invention further provides the use of a fermentation product of the invention as a human food product. In another embodiment, the present invention provides a human food product comprising a fermentation product of the invention. In a further embodiment, the present invention provides a human food product of the invention wherein at least 10%, in particular at least 20%, more in particular at least 30% of the protein content is derived from the fermentation product of the invention. More in particular at least 40%, 50%, 60%, or 70%. In a preferred embodiment, at least 80%, more preferably at least 90%. The present invention further provides methods for producing a human food product, the method comprising mixing animal feed ingredients with the fermentation product of the invention to obtain a human food product.

### Recovery and Recycling of Carbon Dioxide

Further according to the invention, the fermentation of the C1 compound with the methylotrophic microorganism further produces carbon dioxide, which is at least partially recovered and recycled to the reducing step. Carbon dioxide from the fermentation process is sometimes referred to herein as an internal source of carbon dioxide or endogenous CO₂, while remaining carbon dioxide for use in the reduction process may be referred to herein as an external source of carbon dioxide or exogenous CO₂. External sources of CO₂ comprise, but are not limited to, natural gas or derived from an organic source. Recovery and recycling of carbon dioxide from the fermentation process to the carbon dioxide reduction process may be effected by transporting the gas effluent from the fermentation vessel to the carbon dioxide reduction process. In an alternative, preferred, embodiment, the recovering and recycling comprises absorbing carbon dioxide onto a suitable adsorbent followed by the release of the adsorbed carbon dioxide therefrom, e.g. by heat or pressure treatment. In one embodiment, carbon dioxide from the fermentation process is absorbed to an adsorbent and then released and introduced in the carbon dioxide reduction process alone or together with an external carbon dioxide source. In another particular embodiment, gas effluent from the fermentation vessel (comprising carbon dioxide) is mixed with an external carbon dioxide source, after which carbon dioxide from the mixed carbon dioxide sources is adsorbed to an adsorbent and then released and introduced in the carbon dioxide reduction process. Adsorption and release of carbon dioxide can be effected by so-called carbon dioxide scrubbers. Different carbon dioxide scrubber technologies are known and can be used in the present invention. For example, these may be based on amine scrubbing, water scrubbing, carbon dioxide binding minerals and zeolites, sodium hydroxide, lithium hydroxide, activated carbon and metal-organic frameworks. In a particular embodiment, the present methods comprise the use of an amine scrubber for adsorbing and releasing carbon dioxide. In another particular embodiment, the present methods use metal-organic frameworks for adsorbing and releasing carbon dioxide.

Preferably, the recovering of carbon dioxide from the gaseous effluent of the fermentation process is performed under an increased pressure, i.e. a pressure above the atmospheric pressure. This has been found to increase carbon dioxide recovery. In a particular embodiment, the recovering of carbon dioxide is performed at a pressure of 2 bar or more, in particular 5 bar or more, more in particular 10 bar or more. In another particular embodiment, the recovering of carbon dioxide is performed in a pressure range of 2-200 bar, in particular 2 to 100 bar, more in particular 2 to 80 bar.

Performing both the fermentation process and the recovery of carbon dioxide at increased pressure has been found to positively affect the fermentation efficiency and the carbon dioxide recovery efficiency. In addition, the overpressure in the fermentation and carbon dioxide recovery processes can easily be interlinked, providing a straightforward and energy efficient way to increase efficiency during both fermentation and CO₂ recovery, particularly CO₂ adsorption. For example, the increased pressure during fermentation increases the pressure on the gas effluent from the fermentation process as well. This increase pressure can be carried over, partially or completely, to the location of carbon dioxide adsorption, thereby improving CO₂ recovery efficiency as well. In a particular embodiment, the fermentation and the recovering of carbon dioxide is performed at a pressure of 2 bar or more, in particular 5 bar or more, more in particular 10 bar or more. In another particular embodiment, the fermentation and the recovering of carbon dioxide is performed in a pressure range of 2 to 200 bar, in particular 2 to 50 bar, more in particular 2 to 20 bar. The pressure at fermentation and recovering of carbon dioxide from the fermentation effluent may be the same or may be different. In a particular embodiment, the fermentation and the recovering of carbon dioxide is performed at the same pressure and the pressure is 2 bar or more, in particular 5 bar or more, more in particular 10 bar or more. In another particular embodiment, the fermentation and the recovering of carbon dioxide is performed at the same pressure in a pressure range of 2 to 200 bar, in particular 2 to 50 bar, more in particular 2 to 20 bar.

### System and Apparatus

As disclosed herein before, in one aspect, the invention provides a system for performing the methods of the invention. In particular, the system comprises a reduction vessel, a fermentation vessel and a carbon dioxide recovering unit, which have been adapted to perform the methods of the invention. In particular, the system comprises (a) a reduction vessel suitable for reducing carbon dioxide to a C1 compound and (b) a fermentation vessel that is linked to (c) a carbon dioxide recovering unit, such that the carbon dioxide recovering unit can recover carbon dioxide from an effluent of the fermentation vessel. The system may be further adapted to transport carbon dioxide from the carbon dioxide recovering unit to the reduction vessel. Therefore, in a particular embodiment, the present invention provides a system comprising:
a reduction vessel for reducing carbon dioxide to a C1 compound;
a fermentation vessel for culturing a methylotrophic microorganism to convert the C1 compound to a fermentation product; and
a carbon dioxide recovering unit connected to said fermentation vessels to recover at least part of the carbon dioxide produced in the fermentation vessel.

In another embodiment, the invention provides an apparatus for performing the methods of the invention. In particular an apparatus specifically adapted for performing the methods of the invention. In a further embodiment, the present invention provides an apparatus for producing a fermentation product according to the method of any one of the preceding claims comprising:
a reduction vessel for reducing carbon dioxide to a C1 compound;
a fermentation vessel for culturing a methylotrophic microorganism and connected to said first vessel via a duct that allows to transport the C1 compound to the fermentation vessel;
a carbon dioxide recovering unit connected to said reduction and fermentation vessels that allows to recover at least part of the carbon dioxide produced in the fermentation vessel and to recycle said recovered carbon dioxide to the reduction vessel.

In a particular embodiment, the reduction vessel is adapted for the reduction of carbon dioxide to a water soluble, preferably water miscible, C1 compound, in particular methanol, formaldehyde, formic acid, formate or combinations thereof. In a preferred embodiment, the reduction vessel is adapted for the reduction of carbon dioxide to methanol. Suitable reduction vessel techniques, e.g. anodes, cathodes and catalysts, are known to the skilled person to adapt the vessel towards reduction to particular C1 compounds, such as methanol. References provided in relation thereto are mentioned above. In another particular embodiment, the reduction vessel is adapted to enter gaseous carbon dioxide in an aqueous medium, such that when carbon dioxide is reduced to a water soluble C1 compound, the aqueous medium with water soluble C1 compound can be collected through an exit of the reduction vessel.

In another particular embodiment, the reduction vessel comprises an exit to collect a gaseous effluent that has been enriched in oxygen originating from the reduction process. The system or apparatus may further comprise a duct to transport oxygen from the reduction vessel to the fermentation vessel. The duct may further comprise a storage volume to buffer or store oxygen from the reduction vessel until it is required in the fermentation vessel.

In a further embodiment, the apparatus further comprises an electrolysis vessel suitable for the electrolysis of water, wherein the electrolysis vessel comprises a duct to transport hydrogen generated from water electrolysis to the reduction vessel and wherein the reduction vessel is suitable for the hydrogenation of carbon dioxide to a C1 compound. In a further embodiment, system or apparatus further comprises a duct to transport oxygen generated from water electrolysis in the electrolysis vessel to the fermentation vessel.

The fermentation vessel of the present invention may further comprise an exit for collecting at least part of the fermentation culture. In a further embodiment, the apparatus or system further comprises a fermentation product processing unit to enrich or purify the fermentation product from the fermentation culture. The fermentation product processing unit may further comprise a duct to transport products that have not been withheld as enriched or purified fermentation product back to the fermentation vessel. The fermentation product processing unit may comprise one or more centrifuges and/or filters to separate the fermentation product from other products in the fermentation culture. Preferably, the fermentation product processing unit uses microfiltration to separate microorganisms from the remainder of the fermentation culture.

1. A method for producing a fermentation product, which method comprises:
reducing carbon dioxide to a C1 compound,
contacting at least a portion of said C1 compound with a culture comprising a methylotrophic microorganism,
fermenting said C1 compound with said methylotrophic microorganism to produce said fermentation product,
wherein the fermentation of the C1 compound with said methylotrophic microorganism further produces carbon dioxide, which is at least partially recovered and recycled to the reducing step.

2. The method according to claim 1, wherein the carbon dioxide reduction process co-generates oxygen and wherein the oxygen is used to maintain aerobic conditions in the fermentation process.

3. The method according to claim 1, wherein the carbon dioxide reduction process comprises electrolysis of water to oxygen and hydrogen, wherein the hydrogen is used for hydrogenation of carbon dioxide and the oxygen is used to maintain aerobic conditions in the fermentation process.

4. The method according to claim 1, wherein the carbon dioxide reduction process comprises electrochemical reduction of carbon dioxide.

5. The method according to claim 1, wherein said C1 compound is soluble in water.

6. The method according to claim 1, wherein said C1 compound is selected from methanol, formaldehyde, formic acid, and formate, and combinations thereof.

7. The method according to claim 1, wherein said C1 compound is methanol.

8. The method according to claim 3, comprising electrolysis of water to hydrogen and oxygen, wherein
the hydrogen is used for the hydrogenation of carbon dioxide to methanol, and
the oxygen is used to maintain aerobic conditions during the fermentation of the methanol with the methylotrophic microorganism; and
wherein the fermentation produces carbon dioxide which is at least partially recovered and recycled to the hydrogenation process.

9. The method according to claim 4, comprising electrochemical reduction of carbon dioxide to methanol, wherein oxygen is co-generated, and wherein the oxygen is used to maintain aerobic conditions during the fermentation of the methanol with the methylotrophic microorganism; and
wherein the fermentation produces carbon dioxide which is at least partially recovered and recycled to the electrochemical reduction process.

10. The method according to claim 9, wherein the fermenting as well as the recovering of carbon dioxide is performed at an increased pressure.

11. The method according to claim 1, wherein said methylotrophic microorganism is from the group consisting of Methylomonas, Methylobacter, Methylococcus, Methylosinus, Methylocyctis, Methylomicrobium, Methanomonas, Methylophilus, Methylobacillus, Methylobacterium, Hyphomicrobium, Xanthobacter, Bacillus, Paracoccus, Nocardia, Arthrobacter, Rhodopseudomonas, Pseudomonas, Candida, Hansenula, Pichia, Torulopsis, and Rhodotorula.

12. The method according to claim 1, wherein said fermentation product comprises a carbon backbone that is five carbons or longer.

13. The method according to claim 1, wherein said fermentation product is selected from an enzyme, an antibiotic, a chemical, an amino acid, a protein, a plant biostimulant, a growth enhancer, a probiotic, a prebiotic, a biofertilizer, a food, a feed, a vitamin, a lipid, a bioplastic, a polysaccharide, biomass, a bioceutical or a pharmaceutical; preferably wherein said fermentation product is a protein.

14. An apparatus for producing a fermentation product according to the method of claim 1 comprising:
a reduction vessel for reducing carbon dioxide to a C1 compound;
a fermentation vessel for culturing a methylotrophic microorganism and connected to said first vessel via a duct that allows to transport the C1 compound to the fermentation vessel;
a carbon dioxide recovering unit connected to said reduction and fermentation vessels that allows to recover at least part of the carbon dioxide produced in the fermentation vessel and to recycle said recovered carbon dioxide to the reduction vessel.

15. The apparatus according to claim 14, further comprising an electrolysis vessel for electrolysis of water, wherein the electrolysis vessel comprises a duct to transport hydrogen generated from water electrolysis to the reduction vessel, and a duct to transport oxygen generated from water electrolysis to the fermentation vessel.
